(19) United States
(12) Patent Application Publication
(10) Pub. No.: US 2025/0295317 A1
(43) Pub. Date: Sep. 25, 2025

(54) BLOOD PRESSURE MEASURING APPARATUS CAPABLE OF ESTIMATING ARTERIOSCLEROSIS

(71) Applicant: MICROLIFE CORPORATION, Taipei City (TW)

(72) Inventor: Chia-Ming LIN, Taipei City (TW)

(21) Appl. No.: 19/231,904

(22) Filed: Jun. 9, 2025

Related U.S. Application Data
(63) Continuation of application No. 16/794,879, filed on Feb. 19, 2020.

(30) Foreign Application Priority Data
Feb. 20, 2019 (TW) 108105549

(51) Int. Cl. A61B 5/022; A61B 5/0225

(52) U.S. Cl. CPC A61B 5/02225; A61B 5/0225

(57) ABSTRACT

A blood pressure measuring apparatus with a cuff comprises a cuff, an inflation unit, a deflation unit, a pressure sensor, a signal record and storage unit, and an operation and analysis unit. The operation and analysis unit is used to control the inflation unit and the deflation unit to increase the pressure within the cuff to a first pressure, maintain the pressure at the first pressure for a specified interval, then increase the pressure to a second pressure, and decrease the pressure. The blood pressure measurement is accordingly finished. The operation and analysis unit calculates an arteriosclerosis index based on a pulse waveform signal when the adjustable pressure unit maintains the pressure at the first pressure.

### CROSS-REFERENCE TO RELATED APPLICATION

This application is a continuation of U.S. application Ser. No. 16/794,879 filed on Feb. 19, 2020, which claims priority from Taiwan Patent Application No. 108105549 filed on Feb. 20, 2019, both of which are hereby incorporated herein by reference in their entireties.

### BACKGROUND OF THE INVENTION

### Field of the Invention

The invention relates to a blood pressure measuring apparatus capable of estimating arteriosclerosis.

### Description of Related Art

In general, an electronic sphygmomanometer is used to measure arterial blood pressures. The cuff of the sphygmomanometer for sensing blood pressures is inflated and deflated after being wrapped around an upper arm or a wrist of a user. When the cuff is pressurized by inflation and depressurized by deflation, the volumes of pressurized blood vessels accordingly change. Thus, the variation in the amplitudes of the cuff changes is used to calculate blood pressures. Such a technique is called as an oscillometric method. Detailedly speaking, during the measurement of the blood pressures of an upper arm or a wrist, the cuff is inflated till it reaches a certain pressure which is far higher than an averaged systolic blood pressure (generally 30-50 mmHg over the averaged systolic blood pressure) and the inflation is stopped. Afterward, the cuff begins to be deflated for depressurization. When the pressure of the cuff is reduced to a certain level, blood just flows trough vessels. Oscillatory waves propagate through a conduit and reach a pressure sensor. Therefore, the pressure sensor is able to sense and measure pressures within the cuff and the variation thereof.

The electronic sphygmomanometer is expectedly used to measure the systolic blood pressure (SBP), diastolic blood pressure (DBP), heart rate, and so on. Additionally, it is desired to have more functions such as the detection of an arteriosclerosis level and the occurrence of atrial fibrillation.

U.S. Pat. Nos. 6,659,958 and 6,786,872 disclosed an electronic sphygmomanometer capable of measuring an augmentation index (AI). The augmentation index is used for estimating the arteriosclerosis level. After the pressure of the cuff is depressurized to reach a certain pressure value (under a systolic blood pressure), the conventional electronic sphygmomanometer may maintain at the certain pressure value for several or tens seconds. During the interval, it may measure and calculate the incident component and reflective component of a pulse wave. The foregoing augmentation index represents a ratio of the reflective component of the pulse wave to the incident component of it. If aortas are more hardened, the reflective component of the pulse wave on the aortas accordingly get more higher, and consequently a ratio representing the augmentation index is increased. Referring to the flow chart as shown in FIG. 5 of U.S. Pat. No. 6,786,872, it appears that the cuff is inflated till reaches a certain pressure which is far higher than an averaged systolic blood pressure, and then begins to be deflated for depressurization. After the pressure of the cuff is depressurized to reach a predetermined pressure value, it may maintain at the value for several or tens seconds. In the meanwhile, the measurement of general pressure values is finished and they are displayed on a screen. Afterward, the steps of measuring and calculating the augmentation index are performed.

In order to ensure the accuracy of the blood pressure measurement and arteriosclerosis estimating index, the present application provides an a blood pressure measuring apparatus capable of estimating arteriosclerosis and a method using the apparatus to estimate arteriosclerosis.

### SUMMARY OF THE INVENTION

The present application provides a blood pressure measuring apparatus capable of correctly measuring blood pressures and estimating an arteriosclerosis index. It analyzes and obtains characteristic values from the pulse waveform signals of a pressure oscillometric waveform. An arteriosclerosis index such as an augmentation index (AI) or augmentation pressure (AP) is calculated and derived from the characteristic values.

Thus, in one embodiment, the present application provides a blood pressure measuring apparatus capable of estimating arteriosclerosis. The apparatus comprises: a cuff; an inflation unit pressurizing the cuff; a deflation unit depressurizing or discharging the cuff; a pressure sensor used for sensing at least one oscillometric waveform of pressure variation within the cuff; a signal record and storage unit storing at least one pulse waveform signal within the oscillometric waveform; and an operation and analysis unit used to control the inflation unit and the deflation unit to pressurize the cuff till a first pressure and maintain at the first pressure for a specified interval and then pressurize the cuff to a second pressure, and depressurize or discharge the cuff so as to complete blood pressure measurement; wherein the operation and analysis unit calculates an arteriosclerosis index based on a plurality of characteristic values derived from the pulse waveform signal when the cuff is maintained at the first pressure.

In another embodiment, the plurality of characteristic values comprise a first magnitude value of a first peak at an incident component of the pulse waveform signal and a second magnitude value of a second peak at a reflective component of the pulse waveform signal. The arteriosclerosis index is a ratio of the first magnitude value to the second magnitude value.

In another embodiment, the plurality of characteristic values further comprise a third magnitude value of a lowest valley at the pulse waveform signal. The arteriosclerosis index is a ratio of a difference deducting the third magnitude value from the second magnitude value to a difference deducting the third magnitude value from the first magnitude value.

In another embodiment, the plurality of characteristic values further comprise a pulse pressure (PP). The arteriosclerosis index is a ratio of a difference deducting the second magnitude value from the first magnitude value to the pulse pressure.

In another embodiment, the arteriosclerosis index is difference deducting the second magnitude value from the first magnitude value.

In another embodiment, the deflation unit is an adjustable deflation valve which adjusts a depressurized interval based on a heart rate.

In another embodiment, the operation and analysis unit obtains the first and second magnitude values based on a fourth derivative of a function representing the pulse waveform signal.

In another embodiment, the operation and analysis unit obtains the pulse waveform signal by averaging a plurality of pulse waveforms within a plurality of heartbeat periods and stores the pulse waveform signal in the signal record and storage unit.

In another embodiment, the pulse waveform signal acting as a biometric is used to identify a user operating the apparatus.

The present application further provides a method for estimating arteriosclerosis using a blood pressure measuring apparatus. The method comprises: inflating the cuff by the inflation unit after a start of blood pressure measurement so that the cuff is pressurized; confirming whether a pressure of the cuff reaches a first pressure during an inflation process; continuing inflating the cuff till the pressure of the cuff reaches the first pressure; stopping inflation and maintaining the pressure of the cuff at the first pressure for a specified interval; capturing a pulse waveform for at least one heartbeat period during the specified interval to obtain a pulse waveform signal; deriving a plurality of characteristic values from the pulse waveform signal; and calculating an arteriosclerosis index based on the plurality of characteristic values. Afterward, the cuff is pressurized till a second pressure, and then gradually depressurized and simultaneously blood pressure values are measured.

### BRIEF DESCRIPTION OF THE DRAWINGS

In order to sufficiently understand the essence, advantages and the preferred embodiments of the present invention, the following detailed description will be more clearly understood by referring to the accompanying drawings.

FIG. 1 is a function block diagram of a blood pressure measuring apparatus capable of estimating arteriosclerosis in accordance with an embodiment of the present application;

FIG. 2 is a function block diagram of the signal processing unit of the blood pressure measuring apparatus in accordance with an embodiment of the present application;

FIG. 3 illustrates a schematic diagram showing the variation in the pressure of the cuff of the blood pressure measuring apparatus relative to time in accordance with an embodiment of the present application;

FIG. 4 illustrates a schematic diagram of the pulse waveform signal in accordance with an embodiment of the present application;

FIGS. 5A and 5B are schematic diagrams showing the variation in the arteriosclerosis index relative to age in accordance with an embodiment of the present application;

FIG. 6 illustrates a flow chart diagram of estimating arteriosclerosis using the blood pressure measuring apparatus in accordance with another embodiment of the present invention.

### DETAILED DESCRIPTION OF THE INVENTION

The following description shows the preferred embodiments of the present invention. The present invention is described below by referring to the embodiments and the figures. Thus, the present invention is not intended to be limited to the embodiments shown, but is to be accorded the principles disclosed herein. Furthermore, that various modifications or changes in light thereof will be suggested to persons skilled in the art and are to be included within the spirit and purview of this application and scope of the appended claims.

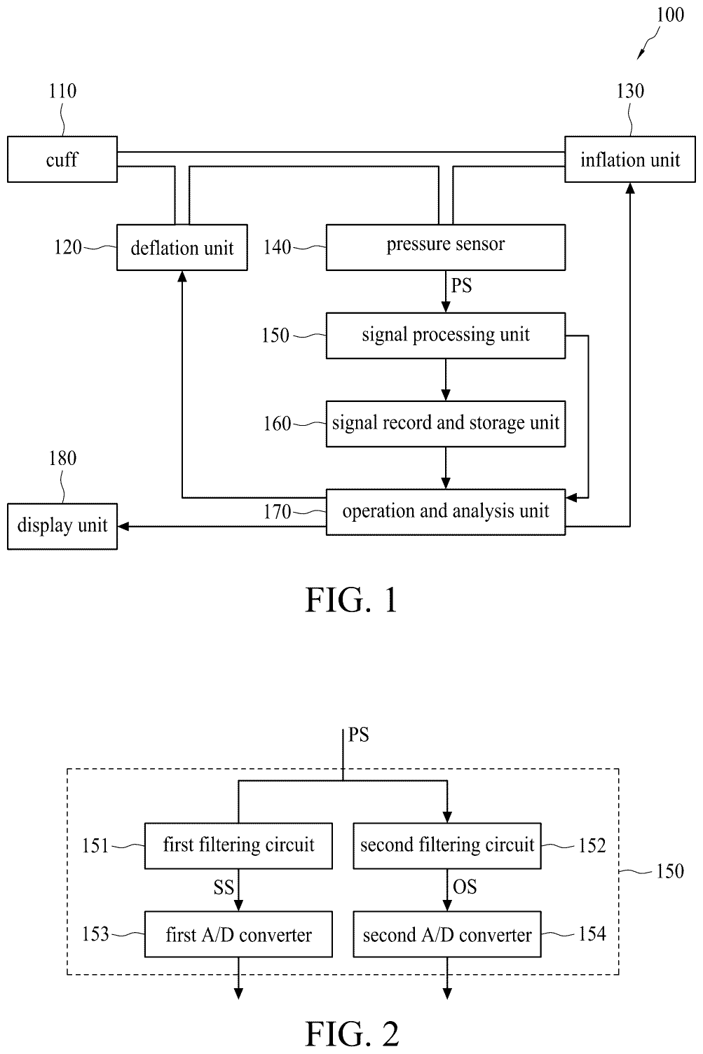

FIG. 1 is a function block diagram of a blood pressure measuring apparatus capable of estimating arteriosclerosis in accordance with an embodiment of the present application. A blood pressure measuring apparatus 100 comprises a cuff 110, a deflation unit 120, an inflation unit 130, a pressure sensor 140, a signal processing unit 150, a signal record and storage unit 160, an operation and analysis unit 170, and a display unit 180. In this embodiment, a method for measuring blood pressure in use is directed to deflation-type measurement. A person skilled in the art may know that a method for measuring blood pressure is also directed to inflation-type measurement.

The pressure of the cuff 110 can be adjusted by the deflation unit 120. In this embodiment, the deflation unit 120 is a deflation valve used to open or close a valve or an adjustable deflation valve, preferable linear valve, used to adjust the opening level of a valve. The inflation unit 130 can inflate the cuff 110. For example, a MEMS pump or an air pump fills the cuff 110 with air. The pressure sensor 140 is communicated with the cuff 110 and the deflation unit 120, and is used to sense the oscillating waveform of the pressure variation in the cuff 110. The pressure sensor 140 can detect a pressure signal PS. The pressure signal PS comprises an oscillating waveform and an air static pressure within the cuff 110, and is outputted to the signal processing unit 150. The signal processing unit 150 conducts signal processing such as filtering and signal conversion on the pressure signal PS. The processed signal is stored or buffered in the signal record and storage unit 160. When the pressure of the cuff 110 is maintained at the first pressure, the operation and analysis unit 170 calculates an arteriosclerosis index according to the pulse waveform signal. The calculation of the arteriosclerosis index will be further described below.

As shown in FIG. 2, the signal processing unit 150 of the blood pressure measuring apparatus 100 receives the pressure signal PS, and has a static pressure portion SS through a first filtering circuit 151. The static pressure is that the cuff 110 pressures the air within it. Furthermore, an oscillating pressure portion OS is obtained after the pressure signal PS is through a second filtering circuit 152. The oscillating pressure is that a brachial artery or a wrist artery pressures the air within the cuff 110. The static pressure portion SS and the oscillating pressure portion OS respectively passes a first A/D converter 153 and a second A/D converter 154. They are converted into digital signals from analogous signals, and the two digital signals are outputted to the operation and analysis unit 170. In other embodiments, the first filtering circuit 154 may be moved to the rear of the first A/D converter 153, but it may be changed into a digital filter from an analogous filter.

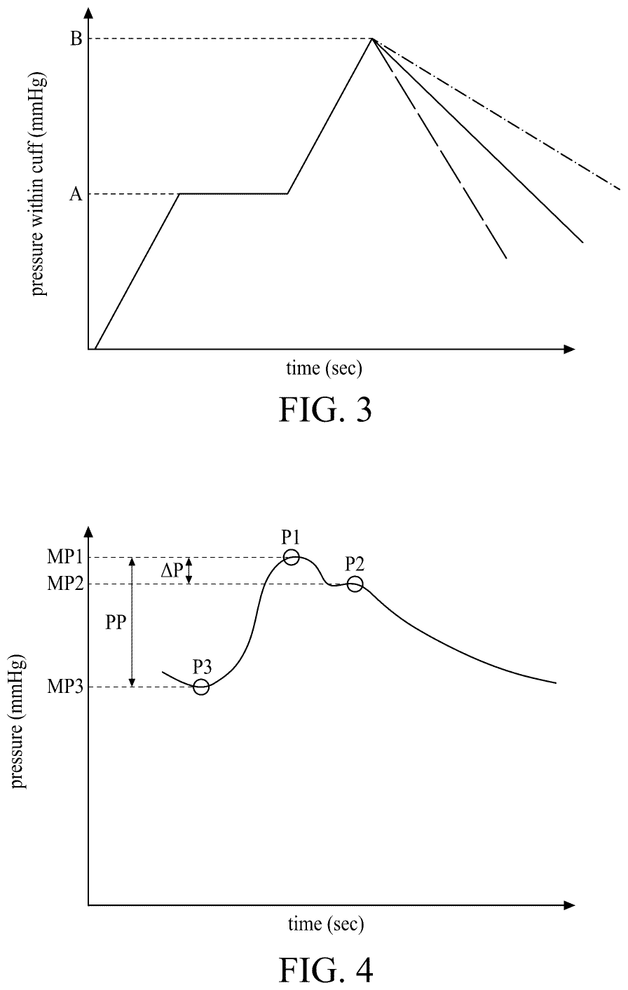

FIG. 3 illustrates a schematic diagram showing the variation in the pressure of the cuff of the blood pressure measuring apparatus relative to time in accordance with an embodiment of the present application. In this embodiment, after a user to be measured wears the cuff 110 and starts blood pressure measurement, the operation and analysis unit 170 controls the inflation unit 130 and the deflation unit 120 to pressurize the cuff 110 till it reaches a first pressure A and then maintain at the first pressure A for a specified interval, preferable 10-15 seconds. The cuff 110 is further pressurized to a second pressure B, and then is depressurized or discharged by the deflation unit 120. The value of the first pressure A is preferably set to be lower than the diastolic blood pressure of the user, and preferably is that the diastolic blood pressure is minus 20 mmHg, but not limited to the application. The second pressure B is preferably set to be higher than the systolic blood pressure of the user. In this embodiment, the diastolic blood pressure of the user may be an average of historical records or one record previously measured and memorized in the signal record and storage unit 160. A person skilled in the art may understand that the historical records are downloaded from an external database to the signal record and storage unit 160. In FIG. 3, the three lines behind the second pressure B respectively represent various rates for depressurizing or discharging the cuff 110. The rate is increased or decreased or a flow rate is adjusted according to the pulse waveform signal (e.g. heartbeat) of the user.

In other embodiments, the blood pressure measuring apparatus 100 may use the pulse waveform signals of various users as their physiological characteristics to match the corresponding historical records or data stored in the signal record and storage unit 160 so as to identify who is the current user to be measured. The pulse waveform signal for each of users has unique waveform characteristics. A person skilled in the art would understand that the historical records or data are downloaded from an external database to the signal record and storage unit 160 in a wire or wireless transmission way.

FIG. 4 illustrates a schematic diagram of the pulse waveform signal in accordance with an embodiment of the present application. The pulse waveform signal has an incident component with a first peak P1 and a reflective component with a second peak P2. The first peak P1 of the incident component and the second peak P2 of the reflective component respectively correspond a first magnitude value MP1 (systolic blood pressure) and a second magnitude value MP2 on the longitudinal axis. The difference between the first magnitude value MP1 and the second magnitude value MP2 is ΔP. The pulse waveform signal has a lowest valley P3 which corresponds a third magnitude value MP3 (diastolic blood pressure) on the longitudinal axis. The difference between the first magnitude value MP1 and the third magnitude value MP3 is PP (pulse pressure).

The formulas for the foregoing arteriosclerosis indexes AID are as follows:

$$AID 1 = MP 1 / MP 2 ; or \qquad formula I$$

$$AID 2 = ( MP 2 - MP 3 ) / ( MP 1 - MP 3 ) ; or \qquad formula II$$

$$AD 3 = Δ P / PP ; or \qquad formula III$$

$$AD 4 = Δ P . \qquad formula IV$$

The foregoing arteriosclerosis indexes AID1-AID3 are augmentation indexes (AI) derived from various formulas, and the arteriosclerosis index AID4 is an augmentation pressure (AP).

The pulse waveform signal as shown in FIG. 4 is expressed by a mathematical function. The operation and analysis unit 170 takes a fourth derivative of the function and has the first magnitude value MP1, the second magnitude value MP2, and the third magnitude value MP3.

Further, the operation and analysis unit 170 averages at least one pulse of the pressure variation in the cuff 110 sensed by the pressure sensor 140 to obtain the pulse waveform signal. Detailed speaking, the operation and analysis unit 170 obtains the pulse waveform signal as shown in FIG. 4 by averaging a plurality of pulse waveforms within a plurality of heartbeat periods and stores the pulse waveform signal in the signal record and storage unit 160. In the other embodiments, the plurality of pulse waveforms measured within a plurality of heartbeat periods may be respectively used for calculation as discussed above. Then, the calculated results are averaged to have the arteriosclerosis index. Or, improper pulse waveforms are excluded, and the others are used for calculation and average.

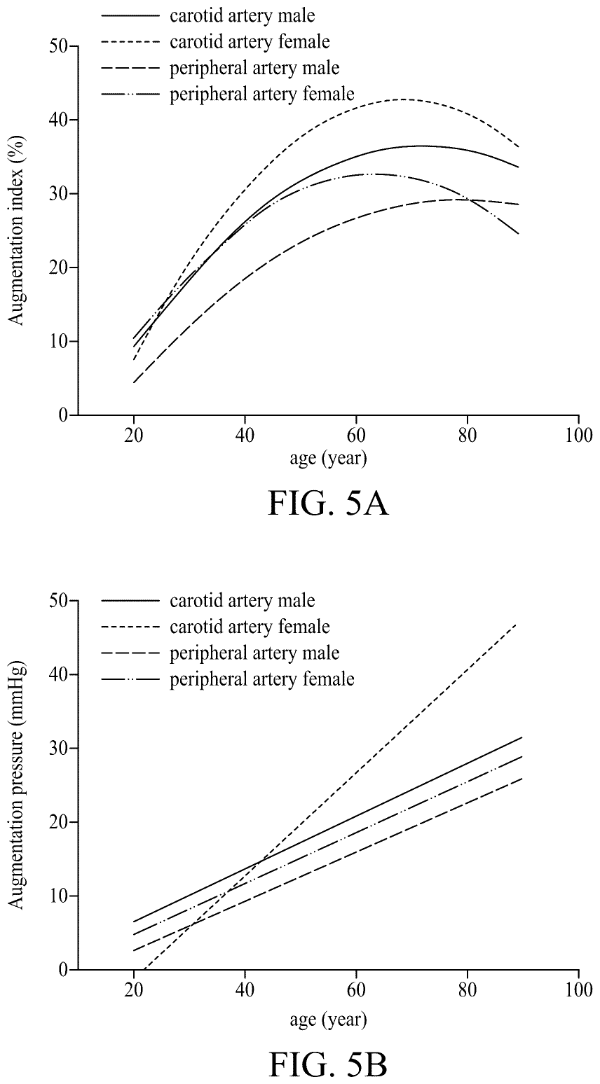

FIGS. 5A and 5B are schematic diagrams showing the variation in the arteriosclerosis index relative to age in accordance with an embodiment of the present application. FIG. 5A illustrates the curves of the augmentation indexes (AI) derived by formula III, and FIG. 5A illustrates the curve of the augmentation pressure (AP) derived by formula IV. The two diagrams can be seen in the thesis written by Francesco Fantin et al. entitled “Is augmentation index a good measure of vascular stiffness in the elderly?” (Age and Ageing 2007; 36:43-48; Published by Oxford University Press on behalf of the British Geriatrics Society.) FIG. 5A shows quadratic curves is representative of quadratic regression augmentation indexes on age in four groups. The augmentation indexes are respectively calculated according to personal pulse waveform signals which are obtained by measuring carotid artery and/or peripheral artery for each of plural males and females. The relationship between augmentation index and age defined by the curves may be stored in the signal record and storage unit 160. When a user uses the blood pressure measuring apparatus 100 to have an augmentation index after measurement, the apparatus can further check the stored relationship between augmentation index and age so as to display an effective age reflected on the arteriosclerosis of the user. Similarly, FIG. 5B shows quadratic curves is representative of quadratic regression augmentation pressures on age in four groups. The augmentation pressures are respectively calculated according to personal pulse waveform signals which are obtained by measuring carotid artery and/or peripheral artery for each of plural males and females.

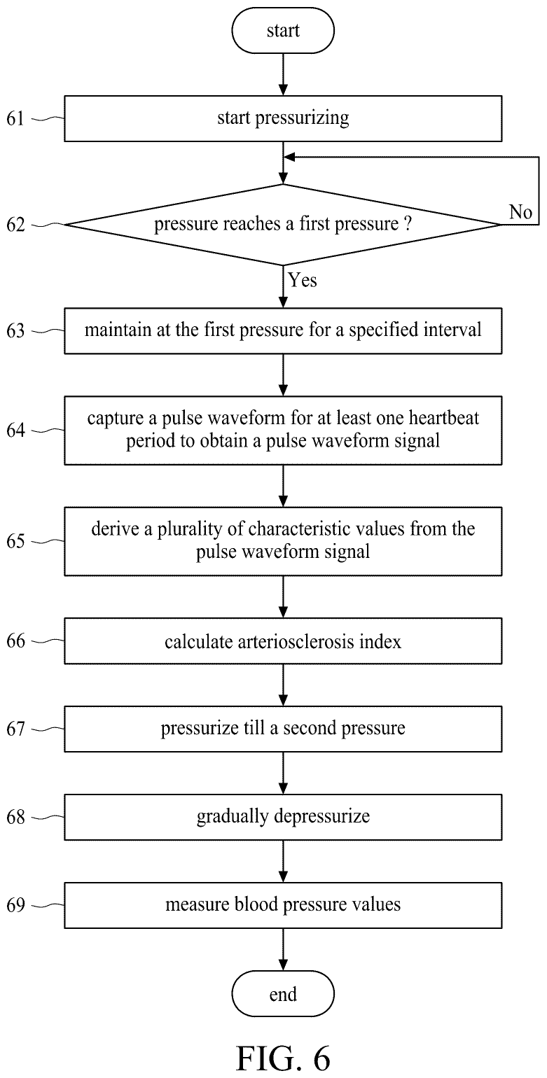

FIG. 6 illustrates a flow chart diagram of estimating arteriosclerosis using the blood pressure measuring apparatus in accordance with another embodiment of the present invention. After a start of blood pressure measurement, the inflation unit 130 proceeds to inflate the cuff 110 so that the pressure within the cuff 110 is increased as shown in Step 61. In the meanwhile, Step 62 is performed. That is, it is to confirm whether the pressure within the cuff 110 reaches a first pressure during the inflation process. If not, the cuff 110 is continuously inflated. On the contrary, the inflation is stopped and goes to Step 63. The pressure of the cuff is maintained at the first pressure for a specified interval. When the first pressure is kept, Step 64 is simultaneously performed to capture a pulse waveform for at least one heartbeat period during the specified interval so as to obtain a pulse waveform signal. A plurality of characteristic values is derived from the pulse waveform signal as shown in Step 65. As discussed above, an arteriosclerosis index is accordingly calculated based on the plurality of characteristic values as shown in Step 66. Afterward, in Step 67, the cuff 110 is pressurized till a second pressure. Step 68 is performed to gradually depressurize the cuff and blood pressure values are simultaneously measured as shown in Step 69.

The foregoing embodiments of the invention have been presented for the purpose of illustration. Although the invention has been described by certain preceding examples, it is not to be construed as being limited by them. They are not intended to be exhaustive, or to limit the scope of the invention. Modifications, improvements and variations within the scope of the invention are possible in light of this disclosure.

What is claimed is:

1. A blood pressure measuring apparatus capable of estimating arteriosclerosis index, comprising:
a cuff worn by a user;
an inflation unit pressurizing the cuff;
a deflation unit discharging the cuff;
a single pressure sensor configured for sensing a pressure oscillometric waveform from the cuff;
a signal processing unit including:
a first filtering circuit obtaining a static pressure portion of the pressure oscillometric waveform;
a second filtering circuit obtaining an oscillating pressure portion of the pressure oscillometric waveform; and
at least one A/D converting the static pressure portion and the oscillating pressure portion into a pulse waveform signal;
a signal record and storage unit storing the pulse waveform signal; and
an operation and analysis unit configured to automatically control the inflation unit to directly pressurize the cuff till a first pressure and stop pressurizing at the first pressure for a specified interval, wherein the cuff does not block blood flow of the user at the first pressure which is lower than a diastolic blood pressure of the user and after the specified interval, immediately pressurize the cuff to a second pressure from the first pressure, wherein the cuff blocks blood flow of the user at the second pressure which is higher than a systolic blood pressure of the user, and then automatically control the deflation unit to discharge the cuff from the second pressure so as to complete deflation-type blood pressure measurement;
wherein the first pressure and the second pressure are detected by the single pressure sensor;
wherein the operation and analysis unit calculates an arteriosclerosis index based on a plurality of characteristic values derived from the pulse waveform signal when the pressure of the cuff is maintained at the first pressure for the specified interval which is 10-15 seconds;
wherein the plurality of characteristic values comprise a first magnitude value of a first peak at an incident component of the pulse waveform signal, a second magnitude value of a second peak at a reflective component of the pulse waveform signal, a third magnitude value of a lowest valley at the pulse waveform signal.

2. The blood pressure measuring apparatus capable of estimating arteriosclerosis index according to claim 1, wherein the arteriosclerosis index is expressed as follows:
$$AID 1 = MP 1 / MP 2$$
where AID1 is an arteriosclerosis index value, MP1 is the first magnitude value, and MP2 is the second magnitude value.

3. The blood pressure measuring apparatus capable of estimating arteriosclerosis index according to claim 1, wherein the arteriosclerosis index is expressed as follows:
$$AID 2 = ( MP 2 - MP 3 ) / ( MP 1 - MP 3 )$$
where AID2 is an arteriosclerosis index value, MP1 is the first magnitude value, MP2 is the second magnitude value, and MP3 is the third magnitude value.

4. The blood pressure measuring apparatus capable of estimating arteriosclerosis index according to claim 1, wherein the plurality of characteristic values further comprise a pulse pressure, and the arteriosclerosis index is expressed as follows:
$$AID 3 = Δ P / PP$$
$$Δ P = MP 1 - MP 2$$
$$PP = MP 1 - MP 3$$
where AID3 is an arteriosclerosis index value, MP1 is the first magnitude value, MP2 is the second magnitude value, and MP3 is the third magnitude value.

5. The blood pressure measuring apparatus capable of estimating arteriosclerosis index according to claim 1, wherein the arteriosclerosis index is expressed as follows:
$$AID 4 = Δ P$$
$$Δ P = MP 1 - MP 2$$
where AID4 is an arteriosclerosis index value, MP1 is the first magnitude value, and MP2 is the second magnitude value.

6. The blood pressure measuring apparatus capable of estimating arteriosclerosis index according to claim 1, wherein the operation and analysis unit obtains the first and second magnitude values based on a fourth derivative of a function representing the pulse waveform signal.

7. The blood pressure measuring apparatus capable of estimating arteriosclerosis index according to claim 1, wherein the deflation unit is an adjustable deflation valve which adjusts a depressurized interval based on a heart rate.

8. The blood pressure measuring apparatus capable of estimating arteriosclerosis index according to claim 1, wherein the pulse waveform signal acting as a biometric is used to identify the user wearing the cuff.

9. The blood pressure measuring apparatus capable of estimating arteriosclerosis index according to claim 8, wherein the second pressure is set to be higher than the systolic blood pressure of the user by 30-50 mmHg.

10. The blood pressure measuring apparatus capable of estimating arteriosclerosis according to claim 9, wherein the first pressure is set to be lower by 20 mmHg than the diastolic blood pressure.

11. The blood pressure measuring apparatus capable of estimating arteriosclerosis index according to claim 10, wherein the diastolic blood pressure of the user is an average of historical records or one record previously measured and memorized in the signal record and storage unit.

12. The blood pressure measuring apparatus capable of estimating arteriosclerosis index according to claim 1, wherein the inflation unit is a MENS pump.
